(19) United States
(12) Patent Application Publication
(10) Pub. No.: US 2024/0027381 A1
(43) Pub. Date: Jan. 25, 2024

(54) BIOSENSORS WITH ONE-DIMENSIONAL CONDUCTING POLYMER SYSTEMS

(71) Applicant: Opteev Technologies, Inc., Baltimore, MD (US)

(72) Inventors: Kallol Mohanta, West Bengal (IN); Lopamudra Bhattacharjee, Kolkata (IN); Soumyo Chatterjee, Kolkata (IN); Anik Sardar, West Bengal (IN); Nyancy Halder, Kolkata (IN); Conrad Bessemer, Millersville, MD (US); Biplab Pal, Ellicot City, MD (US)

(73) Assignee: Opteev Technologies, Inc., Baltimore, MD (US)

(21) Appl. No.: 18/356,713

(22) Filed: Jul. 21, 2023

Related U.S. Application Data
(60) Provisional application No. 63/369,049, filed on Jul. 21, 2022.

(51) Int. Cl. G01N 27/12; G01N 33/569

(52) U.S. Cl. CPC G01N 27/12; G01N 33/56983; G01N 2333/165

(57) ABSTRACT

The present disclosure relates to methods for biosensing using one-dimensional conducting polymer systems. The present disclosure also relates to one-dimensional sensors having substrates coated with conducting polymers such as polyaniline. The geometry of the substrates is configured to maximize the geometrical probability of detecting pathogens, aerosols, etc.

### BACKGROUND

Rapid, accurate, portable, and large-scale diagnostic technologies for the detection of severe acute respiratory syndrome-coronavirus-2 (SARS-CoV-2) are crucial for controlling the spread of coronavirus disease 2019 (COVID-19). Moreover, air-transmissible diseases involving viruses, such as influenza and corona viruses, are responsible for a broad range of adverse health effects, and the risk of exposure to such airborne viruses has increased nowadays due to the utilization of enclosed spaces in many buildings. Airborne viruses are detected, in general, through the bodily fluids of infected hosts. Airborne viruses also can be collected from air either onto a solid surface, or into a liquid medium using a sampler, prior to being assayed for identification and/or quantification. The current gold-standard technology for detecting SARS-CoV-2 is a real-time reverse-transcription polymerase chain reaction (RT-PCR) (E. Z. Ong et al., “A dynamic immune response shapes COVID-19 progression”, Cell Host and Microbe 2020, 27, 879. (doi.org/10.1016/j.chom.2020.03.021), combined with other techniques such as CT scans, enzyme-linked immunosorbent assays (ELISAs), and serological assays. However, critical limitations and challenges are encountered in the practical application of these methodologies.

The RT-PCR method is time-consuming, however, because of the required incubation period, and it is difficult to the detect certain airborne viruses using this method. In view of these limitations, rapid real-time detection of these airborne viruses using alternative bioanalytical techniques is currently needed to control the spread of the pandemic diseases.

During the last 25 years, the majority of biosensor research reported in the relevant literature has been based on the incorporation of bioreceptors into the sensors. Most of the biosensors are self-contained analytical devices with integrated transducer-biorecognition components that convert biochemical events like antigen-antibody interactions to electrical signals. However, the challenges in biosensor development involve: (a) efficient signal capture as a result of biochemical events and their transformation into electrochemical, electrical, or optical signals; (b) improving performance, i.e., increasing sensitivity, shorter response time, reproducibility, and low detection limits even to detect individual molecules; and (c) miniaturization of the biosensing devices. These problems can be addressed by modifying sensing technologies to obtain a high surface-to-volume ratio and noticeable conductivity change.

Conjugated polymer-based biosensors are considered potential alternatives to the above sensors owing to their high selectivity and sensitivity, fast response, cost-effectiveness, simplicity, flexibility, extended self-life, and ease of use. (D. G. Prajapati; B. Kandasubramanian, “Progress in the development of intrinsically conducting polymer composites as biosensors”, Macromolecular Chemistry and Physics, 2019, 220, 1800561. (doi.org/10.1002/macp.201800561)). These types of biosensors are regarded as a three-dimensional network of intrinsically conducting macromolecular wires, able to transport electrical signals. (K. Namsheer; C. S. Rout, “Conducting polymers: a comprehensive review on recent advances in synthesis, properties and applications”, RSC Advances, 2021, 11, 5659. (doi.org/10.1039/DORA07800J)). It also has been reported that polyaniline (PANI) nanocomposites are suitable materials for use in biosensors to achieve synergistic influences, owing to their electro-activity and their ability to be incorporated in biological materials. A single PANI nanowire-based biosensor was developed by Minhee Yun et al., to detect immunoglobulin G (IgG) and myoglobin (Myo), which is one of the cardiac biomarkers. (I. Lee; X. Luo; X. T. Cui; M. Yun, “Highly sensitive single polyaniline nanowire biosensor for the detection of immunoglobulin G and myoglobin,” Biosensors and Bioelectronics, 2011, 26, 3297. doi.org/10.1016%2Fj.bios.2011.01.001). The single PANI nanowires were fabricated via an electrochemical growth method, in which single nanowires were formed between a pair of patterned electrodes. The single PANI nanowires were functionalized with monoclonal antibodies (mAbs) of IgG or Myo via a surface immobilization method. Hao et al. utilized peptide fibres for the fabrication of PANI nanowires that were applied to assemble a biosensor for detecting hepatitis B virus. (Y. Hao; B. Zhou; F. Wang; J. Li; L. Deng; Y-N Liu, “Construction of highly ordered polyaniline nanowires and their applications in DNA sensing”, Biosensors and Bioelectronics, 2014, 52, 422. (doi.org/10.1016/j.bios.2013.09.023)). PANI coated cellulose substrates have drawn immense attention as a sensor material for sensing protein nano particle. Mukheiji et al. disclosed a method of detection and differentiation of bacteria and viruses in samples. (K. Sadani et al., “A point of use sensor assay for detecting purely viral versus viral-bacterial samples”, Sensors and Actuators B: Chemical, 2020, 322, 128562. (doi.org/10.1016/j.snb.2020.128562)). The disclosed method provides the use of impedimetric electro-active PANI immobilized substrate in differentiation of microorganisms based on their interaction with chitosan stabilized silver nanoparticles. Biosensors using conducting polymers such as polyaniline, polypyrrole, poly(3,4-ethylenedioxythiophene), poly(3,4-ethylenedioxythiophene)-polystyrene sulfonate, polyacetylene, poly(3,4-ethylenedioxythiophene), poly (p-phenylene vinylene) and polythiophene, and glutaraldehyde as linker on the disposable paper two-dimensional sensor based on chemoresistivity already have been reported in literature. Djaalab et al., has reported nanomaterials over conducting PANI for electrochemical biosensing of enzyme and proteins. (E. Djaalab; M. E. H. Samar; S. Zougar, R. Kherrat, “Electrochemical biosensor for the determination of amlodipine besylate based on gelatin-polyaniline Iron Oxide biocomposite film”, Catalysis, 2018, 8, 233. (doi.org/10.3390/catal8060233)). Ramanathan et al., and Verghese et al. have reported the immobilization of GOD via manipulation of pore size in conducting polymers respectively to improve loading parameters of the enzyme. (K. Ramanathan; S. S. Pandey; R. Kumar; A. Gulati; A. S. N. Murthy; B. D. Malhotra, “Covalent immobilization of glucose oxidase to poly(o-amino benzoic acid) for application to glucose biosensor”, Journal of Applied Polymer Science, 2000, 78, 662. (doi.org/10.1002/1097-4628(20001017)78:3<662::AID-APP220>3.0.CO;2-T); M. M. Verghese; K. Ramanathan; S. M. Ashraf; B. D. Malhotra, “Enhanced loading of glucose oxidase on polyaniline films based on anion exchange”, Journal of Applied Polymer Science, 1998, 70, 1447. (doi.org/10.1002/(SICI)1097-4628(19981121)70:8%3C1447::AID-APP3%3E3.0.CO;2-4)).

An impedimetric biosensor platform for bio affinity assay was developed by K. Kaur et al., based on a real-time, label-free detection using antimicrobial peptide from class IIa bacteriocins. (H. Etayash; K. Jiang; T. Thundat; K. Kaur, “Impedimetric detection of pathogenic gram-positive bacteria using an antimicrobial peptide from Class IIa bacteriocins,” Analytical Chemistry, 2014, 86, 1693. (doi.org/10.1021/ac4034938)). The biosensor was capable of distinguishing between closely related bacterial strains and was able to detect extremely low concentration of L. monocytogenes with limits of detection as low as 10³ cfu mL⁻1, or the 1 bacterium/μL-a clinically relevant limit. The data analysis algorithm extracted multiple parameters of binding curve kinetics, all of which were essential for analysis of target-peptide interactions in real time.

A. Mulchandani et al. has reported chemiresistive immunosensors based on single polypyrrole (Ppy) nanowire for highly sensitive, specific, label free, and direct detection of viruses. (D. J. Shirale; M. A. Bangar; M. Park; M. V. Yates; W. Chen; N. V. Myung; A. Mulchandani, “Label-free chemiresistive immunosensors for viruses,” Environment Science Technology, 2010, 44, 9030. (doi.org/10.1021/es102129d)). Bacteriophages T7 and MS2 were used as safe models for viruses for demonstration. Ppy nanowires were electrochemically polymerized into alumina template, and single nanowire-based devices were assembled on a pair of gold electrodes by ac dielectrophoretic alignment and anchored using maskless electrodeposition. Anti-T7 or anti-MS2 antibodies were immobilized on single Ppy nanowire using EDC-NHS chemistry to fabricate nano biosensors for the detection of corresponding bacteriophage. The biosensors showed sensitivity with a lower detection limit of 10⁻³ plaque forming unit (PFU) in a 10 mM phosphate buffer.

Different composition ratios of polyaniline (PANI)/poly(ε-caprolactone) (PCL) nanofibers were reported to be produced by electrospinning, and their structure and chemistry have been characterized. (K. Low; C. B. Homer; C. Li; G. Ico; W. Bosze; N. V. Myung; J. Nam, “Composition-dependent sensing mechanism of electrospun conductive polymer composite nanofibers”, Sensors and Actuators B: Chemical, 2015, 207, 235. (doi.org/10.1016/j.snb.2014.09.121)). The PANI/PCL electro spun composite nanofibers were configured in a chemiresistor and subjected to different analytes, including H₂O vapor, NH₃, and NO₂, and H₂O vapor and NO₂ showed a polarity change in sensitivity, having a compositional threshold of PANI-to-PCL ratio.

All the above-mentioned sensors are two dimensional or three-dimensional sensors, and detection has been performed mostly using liquid media sample. This presents a major drawback in the screening of large population on the basis of virus/no virus, i.e., on the basis of the presence or absence of the virus. Moreover, for two-dimensional sensors, the sensitivity of the detection is compromised due to the broad surface area of sensor with respect to the amount of virus that typically falls onto the sensor. To solve this issue, narrowing down the substrate width to the micrometre range might be a good alternative. To date, however, Applicants are unaware of any studies for the detection of biogenic materials with one dimensional sensors having been reported in literature.

Natural fibre can be used as a good alternative for one-dimensional sensors, as there are multiple reports of coating natural fibres with conducting polymer and its composites. In most of the cases, the natural fibres have been used to provide rigidity in conducting polymer-based material, and for the removal of metal contaminants from water.

S. Chakraborty et al. has reported synthesis of polyaniline on jute fibre and a fixed-bed column, and studies were conducted to evaluate performance of a short-chain polymer, polyaniline, synthesized on the surface of jute fibre (PANI-jute) for the removal of hexavalent chromium [Cr(VI)] in an aqueous environment. (P. A. Kumar; S. Chakraborty, “Fixed-bed column study for hexavalent chromium removal and recovery by short-chain polyaniline synthesized on jute fiber”, Journal of Hazardous Materials, 2009, 162, 1086. (doi.org/10.1016/j.jhazmat.2008.05.147)). However, the PANI-jute composite hitherto has not been utilised for virus or other biogenic particle detection. Natural fibres have many advantages such as low cost, low density, renewability etc. Jute is a bast fibre whose scientific name is Corchorus capsularis of Tiliaceae family. India and Bangladesh are top jute producing countries in the world. Jute contains cellulose (64.4%), hemi-cellulose (12%), lignin (11.8%), pectin (0.2%) & 1.1% water content. (M. K. Gupta; R. K. Srivastava; H. Bisaria, “Potential of jute fibre reinforced polymer composites: A Review”, International Journal of Fiber and Textile Research, 2015, 5, 30).

Silk fabric is the one of the strongest natural fibres. It is lightweight, has good mechanical properties and biocompatibility, and can strongly bind to active materials because of an abundance of functional groups, such as carboxyl, amino, and hydroxyl groups. The dissolution of silk protein by ternary solvent (CaCl₂)/C₂H₅OH/H₂O) is exploited to pre-treat silk fabric to increase the specific surface area and the active material loading. The resulting high active material loading and the strong interaction between the active material and the silk solved many problems of poor charging and discharging ability, and low area capacitance of the electrode. (H. Cai et al., “High performance flexible silk fabric electrodes with antibacterial, flame retardant and UV resistance for supercapacitors and sensors”, Electrochimica Acta, 2021, 390, 138895. (doi.org/10.1016/j.electacta.2021.138895)). An improved method for preparation of conductive silk fibroin yarns coated with polyaniline was shown by J. Hong et al., based on in situ polymerization of aniline. (J. Hong; X. Han; H. Shi; L. Jin; J. Yao, “Preparation of conductive silk fibroin yarns coated with polyaniline using an improved method based on in situ polymerization” Synthetic Metals, 2018, 235, 89. (doi.org/10.1016/j.synthmet.2017.12.002)). Electrically conductive and flexible silk fabrics can be prepared by electrostatic self-assembly between silk fibroin and polyaniline or other conducting polymers.

Dimensions, and dimension-related terminology such as “one-dimensional,” “two-dimensional,” “three-dimensional,” etc., are used herein within the context of the macroscopic world. “One-dimensional,” “two-dimensional,” “three-dimensional,” etc., for the purposes of this disclosure, should be considered as physical properties of things as realized by and/or with human senses, and not beyond that. This essentially means that the descriptions and claims presented herein can be explained and achieved with, and are restricted within the realms of classical science.

Disadvantages of 2D Sensors

Within the context of biosensing, in the case of conducting polymer-based chemoresistive sensing of biogenic particles including viruses, bacteria, etc. (where the polymers can include, for example, Polyaniline/Polythiophene and others), interaction occurs via interaction of the functional group (—NH₂/—CHO/—COOH) of conducting polymers having their conductivity destroyed over the small area of attachment to the sensor. For aerosolized viruses, since droplet size is between 0.1 micron to 10 microns, any aerosol falling on the sensor can alter the conductivity of a region which is of maximum 30 micron by 30 micron size as evidenced by the scanning electron microscope (SEM) images shown in FIG. 1.

The left image of FIG. 1 depicts a conducting polymer (PANI) grown in-situ from the synthesis bath on a paper substrate of 300 grams per square meter (gsm) basis weight. The PANI depends on the size of paper fibre which is in this case about 20 microns to about 25 microns in width. Interaction of natural fibres (silk and cellulose, i.e., jute) with polyaniline. The right image of FIG. 1 is an SEM image of viral particles fallen on a substrate. The individual particle is globular and has diameter >100 nm. However, the viral particles aggregate, and the size of the aggregate can be up to 10 microns to 20 microns.

Since the size of biosensors typically is 4 mm² to 10 mm², the maximum number of aerosols from human breathing that can be captured over two to four minutes of exposure from a distance of two to three feet is approximately 3,000. Thus, even in the best probable case, the percentage of the 2D structure over which the aerosols can alter the conductivity is below (3000×30×30/2000×2000), or about 60 percent, considering the sensor size as 2 mm² by 2 mm². Therefore, the kinds of 2D structures that have been reported in the earlier patents and literature are not useful for detection of aerosolized viruses as evidenced from the several experimental results obtained by the Applicant and shown in FIGS. 8 and 12.

The sensitivity of conducting polymer-based sensors depends on multiple factors such as the choice of the substrate base, uniform coating of conducting material on the substrate, etc. These effects are more evident in wider sensors.

On the other hand, another critical aspect and intrinsic challenge of all 2D materials is the broad surface area leading to constant absorption of environmental gases, VOCs, and moisture, which leads to a minute but steady decremental changes in the baseline conductivity of the system upon environmental exposure. Also, control (passivation) of surfaces has been one of the challenges in the fabrication of electronic devices.

Moreover, a most important factor is that, for a very small viral load, viral exposure on a very small area of the substrate cannot produce a substantial change in resistivity. This may be due to the presence of multiple possible parallel conducting paths of the charge carrier within the substrate.

### SUMMARY

In one aspect of the disclosed technology, a sensor in solid-state form for detection of viruses in aerosolized form has been developed by varying the size and geometry of the substrate of the sensor.

In another aspect of the disclosed technology, a virus-detection method using the sensor includes measuring a change in impedance of the sensor upon exposure of the sensor to the virus.

In another aspect of the disclosed technology, the solid-state sensor includes electrically-conducting, polymer-coated natural fibres for detecting aerosolized viruses in a solid-state media.

In another aspect of the disclosed technology, the conducting polymer is Polyaniline (PANI).

In another aspect of the disclosed technology, two-dimensional (2D) sensor substrates were compared with one-dimensional (1D) sensor substrates, and it has been determined that 1D substrates have greater sensitivity than 2D sensors for aerosolized virus detection in solid-state media, consistent with theoretical explanation.

In another aspect of the disclosed technology, modification of sensor geometry showed a change in sensor sensitivity.

In another aspect of the disclosed technology, 1D sensors based on natural fibres such as, silk, jute, etc., have been prepared and it has been observed that change in geometry from single thread (1D) to criss-cross (1D) makes the sensors much more sensitive to aerosolized virus detection.

In another aspect of the disclosed technology, a response time for the 1D substrate is lower than that of the 2D substrate.

In another aspect of the disclosed technology, a detection system based on change of impedance in terms of frequency includes an RC coupling circuit.

In another aspect of the disclosed technology, a sensor for detecting the presence of pathogens incudes a one-dimensional substrate.

In another aspect of the disclosed technology, the sensor further includes a coating on the substrate, the coating including an electrically-conducting polymer.

In another aspect of the disclosed technology, the coating includes polyaniline.

In another aspect of the disclosed technology, the substrate includes cellulose.

In another aspect of the disclosed technology, the substrate includes a single-strand filament.

In another aspect of the disclosed technology, the substrate includes a natural fibre.

In another aspect of the disclosed technology, the substrate includes one of silk thread In another aspect of the disclosed technology, the substrate has a width of less than about 25 microns.

In another aspect of the disclosed technology, the substrate has a width of about 3 microns to about 25 microns.

In another aspect of the disclosed technology, the substrate has a width of about microns to about 25 microns.

In another aspect of the disclosed technology, the substrate has a linear configuration.

In another aspect of the disclosed technology, the substrate has a zig-zag configuration.

In another aspect of the disclosed technology, the substrate has a criss-cross configuration.

In another aspect of the disclosed technology, the substrate includes paper.

In another aspect of the disclosed technology, the substrate includes 300 grams per square meter paper.

In another aspect of the disclosed technology, a method for detecting the presence of airborne pathogens includes providing a one-dimensional sensor; and measuring a change of resistance of the sensor in response to exposure to the pathogens.

In another aspect of the disclosed technology, providing a one-dimensional sensor includes providing a sensor that includes a substrate having a width of about 25 microns or less, and a coating on the substrate. The coating includes an electrically-conducting polymer.

In another aspect of the disclosed technology, a sensor for detecting the presence of pathogens includes a substrate having a width of about 25 microns or less; and a coating on the substrate. The coating includes an electrically-conducting polymer.

In another aspect of the disclosed technology, the coating includes polyaniline.

In another aspect of the disclosed technology, pathogen detection system includes a sensor; and an RC coupling circuit electrically coupled to the sensor and configured to indicate a change of resistance of the sensor based on an output frequency of the RC coupling circuit.

### BRIEF DESCRIPTION OF THE DRAWINGS

The following drawings are illustrative of particular embodiments of the present disclosure and therefore do not limit the scope of the present disclosure. Embodiments of the present disclosure will hereinafter be described in conjunction with the appended drawings, wherein like numerals denote like elements.

FIG. 1 depicts a conducting polymer (PANI) grown in-situ from a synthesis bath on a paper substrate; and a scanning electron microscope image of viral particles that have fallen on a substrate and aggregated;

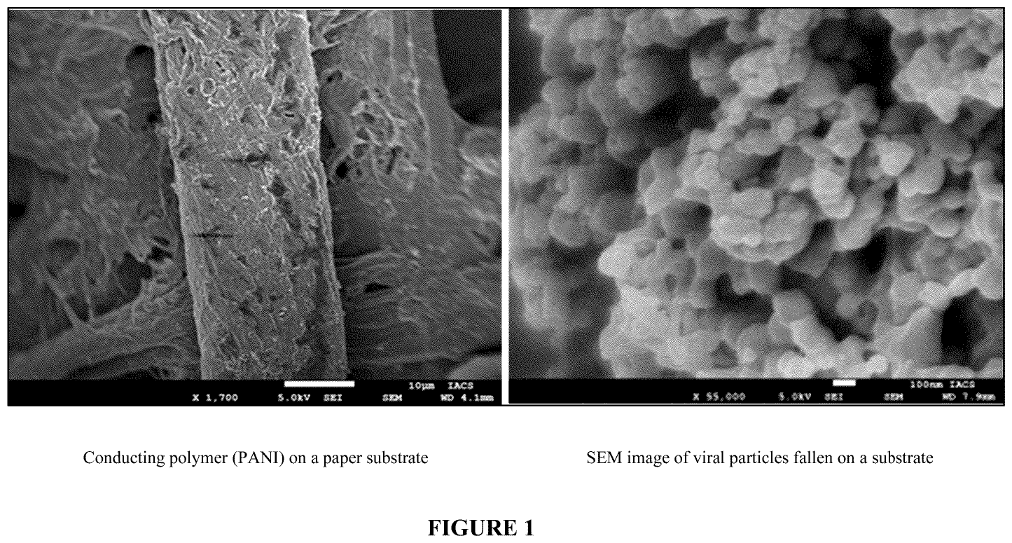

FIG. 2 is a schematic illustration of two sensors, showing the lower number of electronic conducting paths in the sensor having the smaller width.

FIG. 3 shows comparative resistance measurements obtained from sensors having relatively wide and narrow substrates;

FIG. 4 is a table showing various steps in the fabrication process for the sensors shown in FIG. 5;

FIG. 5 is a schematic diagram of different types of 1D and 2D sensors;

FIG. 6 is a simplified circuit diagram of an RC oscillation circuit for determining a change in resistance of a sensor;

FIG. 7 depicts an exemplary test set-up for exposing sensors to airborne viral particles;

FIG. 8 includes graphical representations of the respective responses of a 2D paper-based sensors and a 1D paper-based sensor to exposure to an equivalent number of airborne viral particles;

FIG. 9 includes graphical representations of the responses of 1D sensors comprising silk and jute fibres, respectively, to exposure to airborne viral particles.

FIG. 10 is a diagrammatic illustration showing the chemical interaction of natural fibres in the form of silk and cellulose, i.e. jute, with polyaniline.

FIG. 11 is a graphical representation of the response of a 1D paper sensor to exposure to airborne viral particles, showing the effects of varying to the length of the sensor.

FIG. 12 includes graphical representations of the respective responses of a 2D paper-based sensor and a 1D paper paper-based sensor to exposure to an equivalent number of airborne viral particles.

FIG. 13 is a graphical representation of the response of a 1D sensor comprising silk fibres to exposure to airborne viral particles.

FIG. 14A is a graphical representation of the response of a 1D sensor to exposure to different concentrations of SARS Cov-2 viruses.

FIG. 14B is a graphical representation of the rate of change of the normalized response of the 1D sensor to the exposure to different concentrations of SARS Cov-2 viruses.

### WRITTEN DESCRIPTION

The inventive concepts are described with reference to the attached figures, wherein like reference numerals represent like parts and assemblies throughout the several views. Several aspects of the inventive concepts are described below with reference to example applications for illustration. It should be understood that numerous specific details, relationships, and methods are set forth to provide a full understanding of the inventive concepts. One having ordinary skill in the relevant art, however, will readily recognize that the inventive concepts can be practiced without one or more of the specific details or with other methods. In other instances, well-known structures or operation are not shown in detail to avoid obscuring the inventive concepts.

The inventive concepts are described with reference to the attached figures, wherein like reference numerals represent like parts and assemblies throughout the several views. Several aspects of the inventive concepts are described below with reference to example applications for illustration. It should be understood that numerous specific details, relationships, and methods are set forth to provide a full understanding of the inventive concepts. One having ordinary skill in the relevant art, however, will readily recognize that the inventive concepts can be practiced without one or more of the specific details or with other methods. In other instances, well-known structures or operation are not shown in detail to avoid obscuring the inventive concepts.

Theoretical Advantages of 1D Sensors

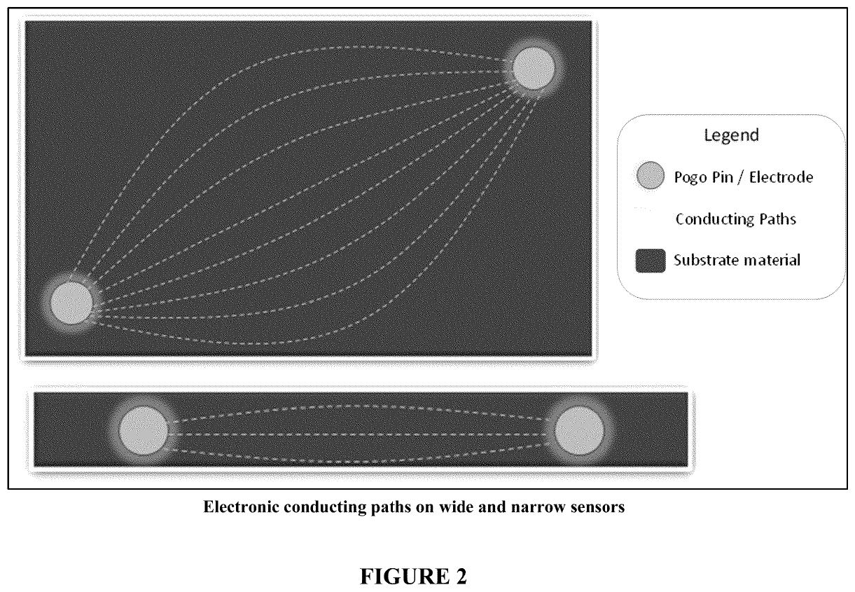

Within a conductor, according to free-electron gas theory, an electron faces no barrier and thus can move in any direction. When a conductor is placed within an electric field, the electrons experience a net force in the opposite direction of the applied field, and move along the direction of force. Although there are many probable free paths, electrons follow linear free paths having larger components in the direction to travel since those free paths result in the least time for the electrons to travel from cathode to anode. Also, is possible for the electrons to travel over multiple mean free paths at one time. In case the mean free path(s) is (are) disrupted, the electrons find alternative (new) mean free paths for conduction. Thus, for any conductor subjected to electrical characterization, multiple probable parallel conduction paths exist between the two electrodes even when the conductor is in a 2D configuration, as shown in FIG. 2. According to the Landauer principle, the electric current through a material is given by:

$$I = - \frac{2 q}{h} \int T ( E ) \cdot M ( E ) \cdot ( {n}_{1} - {n}_{2} ) dE ,$$

where q is the electronic charge, h is the Plank constant, T(E) is the transport probability, M(E) is the modes available and n's are the occupation numbers at energy levels E and (E+dE). (D. Wortmann; H. Ishida; S. Blugel, “Embedded Green-function formulation of tunneling conductance: Bardeen versus Landauer approaches” Physical Review B, 2005, 72, 235113. (doi.org/10.1103/PhysRevB.72.235113). The current (1) depends on the area of the 3D conductor, or the width of conductor for the 2D conductor when the conductor length is constant. Thus, decreasing the width of a 2D conductor, as shown in FIG. 2, decreases the number of electronic conducting paths and thereby increases the resistance while reducing the current.

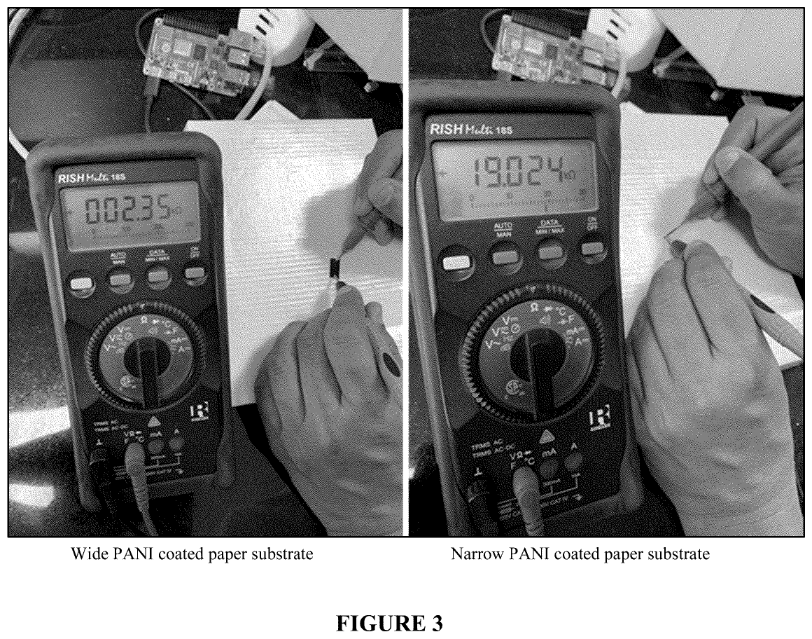

For example, FIG. 3 depicts a resistance measurement of a wide PANI-coated paper substrate on left, and a resistance measurement of a relatively narrow PANI-coated paper substrate on the right, with the narrow substrate showing much higher resistance than the wide substrate. Classically, this means a reduction of mean free path number. (For a quantum mechanical 1D conductor, however, the modes are constant of energy and the transmission probability becomes zero for a conductor which has a length much longer than the electron scattering distance.) As noted above, in the context of the present application, this description remains restricted within classical science boundaries, and “one-dimensional” or “1D” is intended to mean the narrowest two-dimensional (2D) conductor which obeys classical scientific laws. In such a conductor, the number of electronic mean free paths within the conductor would be at a minimum. According to the development described herein, a 2D conductor with a width within the few micrometer range can be achieved, for example, using silk thread as the substrate.

From earlier reports, it is known that the virus particle and biogenic materials bind with PANI (conducting polymer) chains through imine/amine bonds. (R. Borah; A. Kumar; M. K. Das; A. Ramteke, “Surface functionalization-induced enhancement in surface properties and biocompatibility of polyaniline nanofibers”, RSC Advances, 2015, 5, 48971. (doi.org/10.1039/C5RA01809A)). This bond formation withdraws the excess (free) electron from the conjugated electronic pathway of PANI chain and impairs local electronic conductivity. (S. Griggs; A. Marks; H. Bristow; I. McCulloch, “n-Type organic semiconducting polymers: stability limitations, design considerations and applications” Journal of Materials Chemistry C, 2021, 8099. (doi.org/10.1039/D1TC02048J)). This means that virus exposure at a point on the conductor disrupts the electronic path through that point and the enclosing area (about 30 microns for a virus-containing aerosol). Thus, if the number of available electronic paths (including parallel conduction paths between the two electrodes) is at a minimum, destroying some of them immediately causes a drastic drop of conductivity and/or a drastic increase of resistance (about 1.5 to 2 times or greater). Thus, the sensitivity becomes very large and the response time very short (about 10s to 40s, due to the time taken to absorb the aerosol).

Hence, when the conducting polymer based 2D substrates are exposed to aerosolized virus particles, the incidence of the virus particles results in a significant increment of resistance of some of such paths (and not necessarily the least resistive one), thereby leading to drop in conductivity. But due to the parallel geometry of several such resistive paths, significant alteration in the equivalent resistance of the network after virus exposure is no longer a repetitive phenomenon. Restriction of the substrate dimension to 1D resists the formation of multiple parallel conduction paths, and suddenly and appreciably drops down the flow of current upon the same load of virus exposure. Hence, in theory, an almost completely resistive nature should be observed after exposure of the 1D substrate to the virus.

Experimental Verification

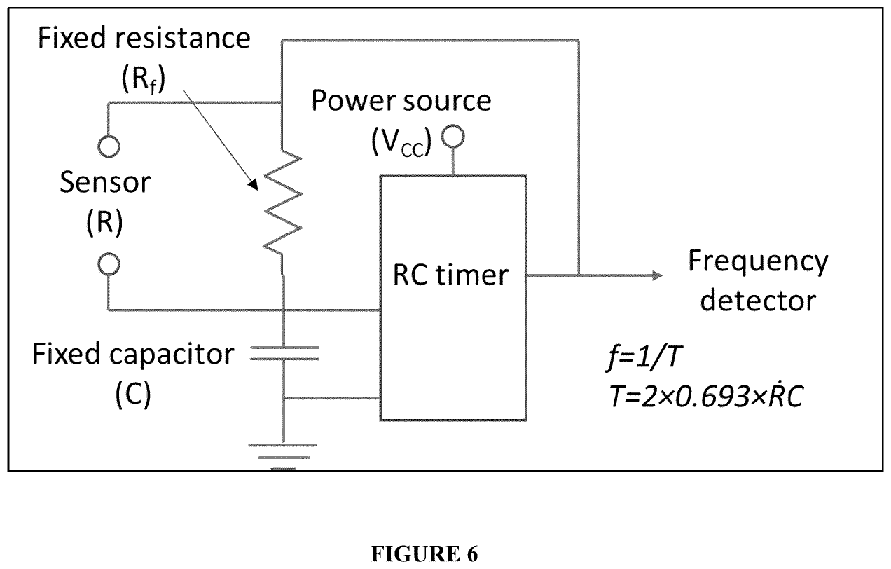

To confirm the above phenomena experimentally, a detection method was designed in which the sensor is coupled with RC-based electronics which provide an output in terms of frequency. In this two-electrode electronic circuit, shown in FIG. 6, the resistance of the sensor and the frequency output from the circuit maintain an inverse relationship with each other.

Sensor Fabrication

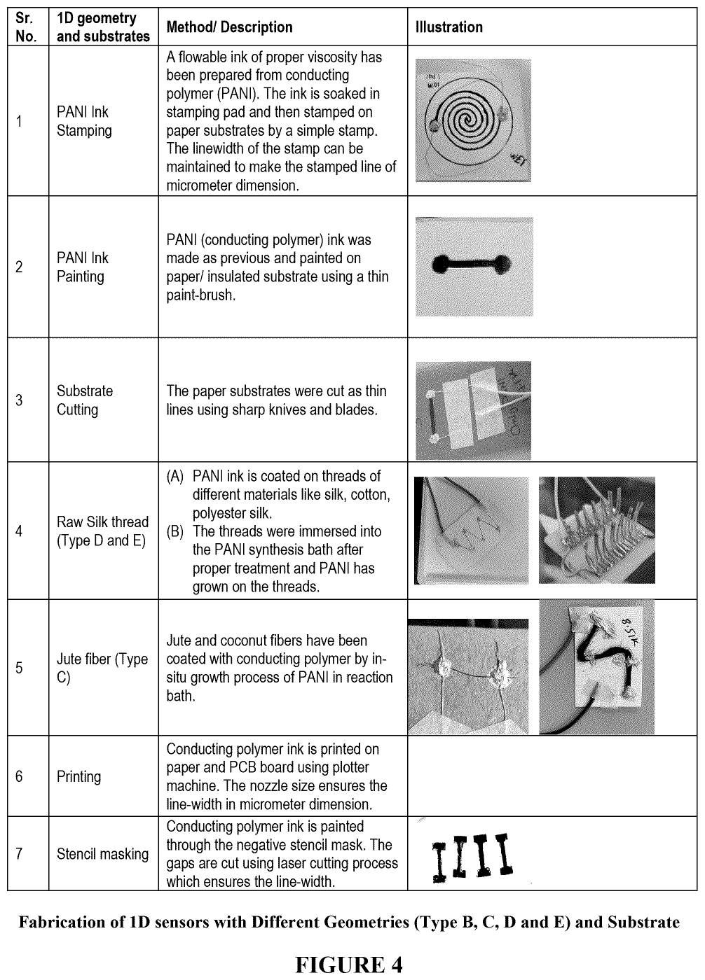

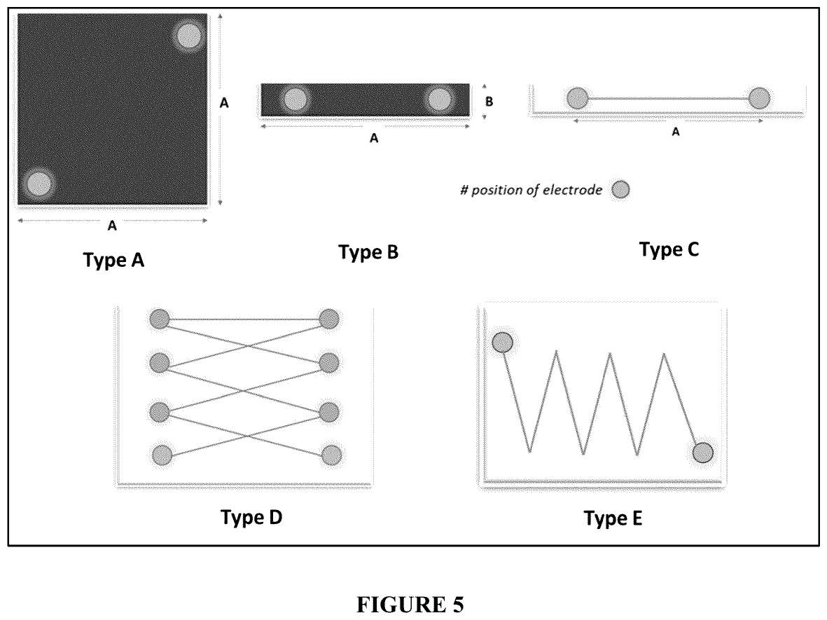

The following sensors, depicted in FIG. 5, were fabricated as follows. The senor designated “Type A” is a 2D sensor, and the other depicted sensors are examples to 1D sensors with different types of geometries. Various steps in the fabrication methodology are documented in the table presented in FIG. 4.

Preparation of Type-A substrate: Polyaniline (PANI) was in-situ synthesized/coated on hot pressed 300 GSM cellulose substrate (Type A) (2D) by oxidative polymerization using 0.1 M Aniline in 1M HCl and ammonium peroxodisulfate (APS) as oxidizing agent (equimolar to aniline). The polymerization reaction was performed in cooled (32° F.-37.5° F.) reactor under continuous stirring. On completion of polymerization each substrate was coated with 2 percent solution of Terephthaldehyde in ethanol.

Preparation of Type-B substrate: Polyaniline (PANI) was in-situ synthesized/coated on hot pressed 300 GSM paper cellulose (Type B) substrate by oxidative polymerization using 0.1 M Aniline in 1M HCl and ammonium peroxodisulfate (APS) as oxidizing agent (equimolar to aniline). The polymerization reaction was performed in a cooled (32° F.-37.5° F.) reactor under continuous stirring. Terephthaldhyde coating was done after polymerization as mentioned for Type A substrate. Then the sensor substrates were cut into a narrow (approximately 1 mm width) piece.

Preparation of Type-C(filament like) substrate: Polyaniline (PANI) was in-situ synthesized/coated on jute and silk thread by oxidative polymerization using 0.1 M Aniline in 1M HCl and ammonium peroxodisulfate (APS) as oxidizing agent (equimolar to aniline). The polymerization reaction was performed in cooled (32° F.-37.5° F.) reactor under continuous stirring followed by coating with 2% Terephthaldehyde solution. The threads were dried at 50° C. in a hot air oven.

Preparation of Type-D (filament like) substrate in criss-cross fashion: PANI coated Type-C substrate was arranged in criss-cross fashion around multiple arrays of pins.

Preparation of Type-E (filament like) substrate in zig-zag fashion: PANI coated Type-C substrate was pasted with silver paste on a PVC board in zig-zag fashion.

Measurement of Resistivity

As discussed above, the exposure of viral particles or biogenic material decreases the conductivity of PANI, i.e., the conducting polymer. This essentially means that the resistance of the sensor increases, and that is the sensing signature of the viral particles or biogenic materials. A detection method has been developed to determine such changes in resistance. The detection method couples the sensor with RC based electronics, to provide an output in terms of frequency.

The sensor resistance is used to generate an oscillation in an RC oscillation circuit as shown, for example, in the simplified circuit diagram of FIG. 6, with the oscillation frequency changing with the sensor resistance. Thus, the change in the resistivity/conductivity of the substrate can be measured from the change in the oscillation frequency. In the setup depicted in FIG. 6, the sensor is connected with the RC oscillator (e.g., RC timer, LMC555) in parallel with a fixed-value resistor (e.g., R=150 kΩ), and an initial frequency (f) is generated based on the sensor resistance (R) which is in parallel with the fixed resistor. The calculation of the frequency f is as follows:

$$f = \frac{1}{0.693 \times 2 \times \dot{R} C}$$

where \dot{R}=R_{f}∥R or

$$\frac{1}{\dot{R}} = ( \frac{1}{{R}_{f}} + \frac{1}{R} ) ,$$

R_{f}=fixed resistance, R=Sensor/substrate resistance.

$$f = \frac{1}{k \times ( {R}_{f} \vert \vert R )} ,$$

Where k=1.386×C

$$f = \frac{\frac{1}{{R}_{f}} + \frac{1}{R}}{k} \\ f = \frac{1}{k \times {R}_{f}} + \frac{1}{k \times R}$$

$$f = 481 + \frac{72.15 \times {10}^{6}}{R} ,$$

for C=0.01 μF and R_{f}=150 kΩ (used in circuit)

$$f = {f}_{0} + \frac{k}{R} ,$$

where f₀=481 Hz and k=72.15×10⁶ F⁻¹

$$f = {f}_{0} + {f}_{x} ( \frac{1}{R} ) ,$$

Where fₓ=is the additional frequency generated by the substrate which is inversely proportional to the change of R.

The addition of the virus particles on the cellulose substrate is indicated by a drop in the output frequency of the sensor.

### Results

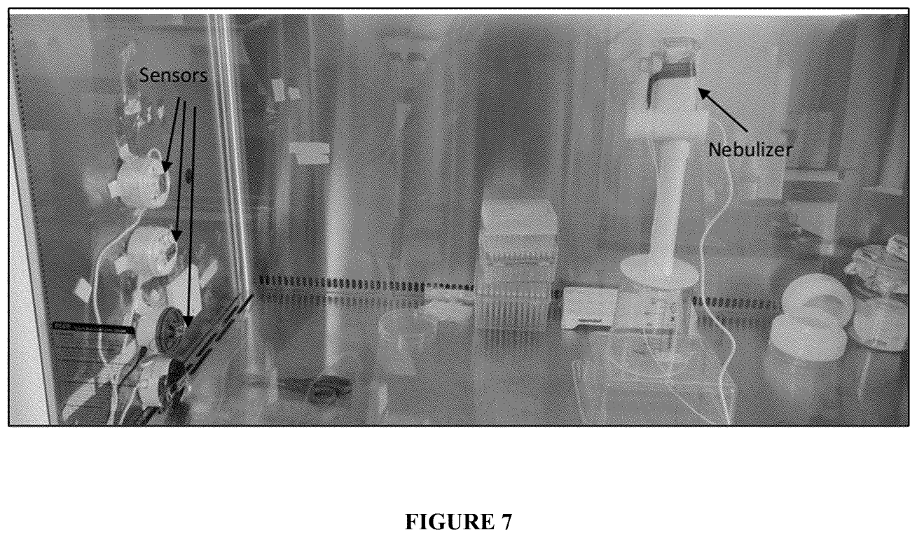

An illustrative, exemplary test set-up is depicted in FIG. 7. In the test set-up, the detection system was arranged in a box, and the sensor was placed on the box using adhesive tape. The connections were taken through wire connectors or pogo pins. A nebulizer containing the viral particles/biogenic materials in a suitable solvent was separated from the sensor by a distance of about two to about three feet.

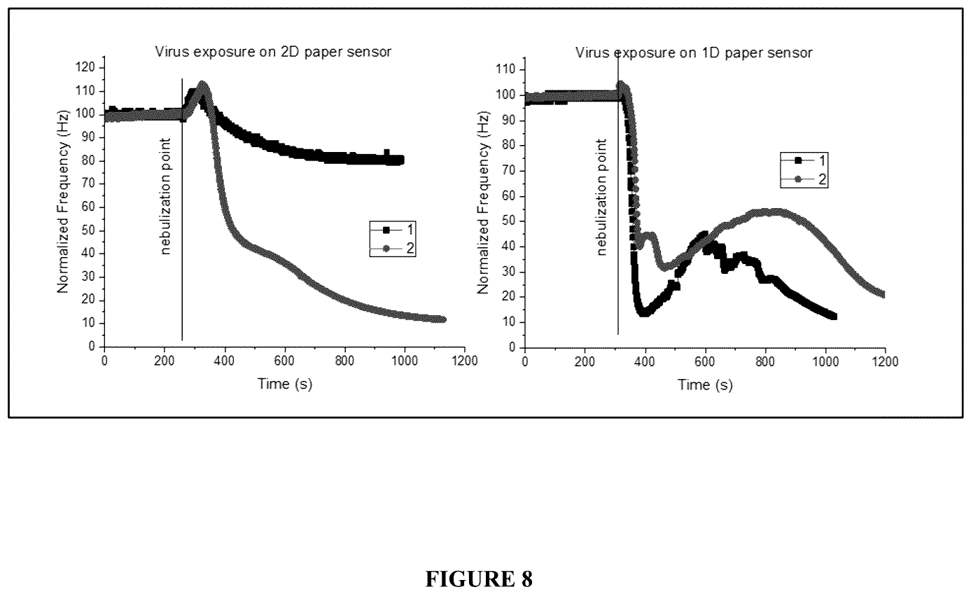

The test results are shown in FIG. 8. In particular, FIG. 8 displays representative data showing a side-by-side comparison of the responses of a 2D paper-based sensor (on the left) and 1D paper-based sensor (on the right) upon exposure to the same number of viral particles (20 k viral particles). FIG. 8 shows that the normalized frequency drops, i.e., the figure of merit value, is higher for the 1D sensor, and the response of the 1D sensor is faster than that of the 2D sensor.

The results thus indicate that a single-strand-filament 1D sensor gives better results over a multistrand-filament 2D sensor in terms of sensitivity. Comparing the response of the 1D sensor and the 2D sensor, the sensitivity has been increased in the 1D sensor, as can be seen clearly from the larger frequency drop. Also, the response time has been decreased, as evidenced from the higher rate of frequency drop in case of the 1D sensor.

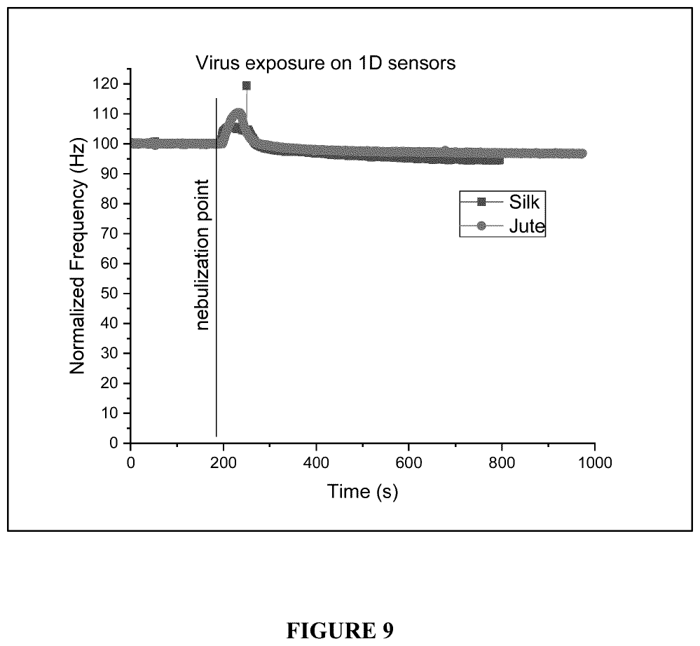

FIG. 9 exhibits responses from a single strand silk substrate with criss-cross geometry (Type D) and a jute fibre in zig-zag geometry (Type E). Although a drop in frequency can be observed, it is not as large as that of the paper sensors. The reason could be the low cross-sectional area available to capture the nebulized viral particles, because even the natural fibres like jute, cotton, silk, etc. and their threads (single or multiple strands) reduce the thickness of the substrate further. Changing the substrate geometry from that of Type C (A×B [B«A] as show % n in FIG. 5) to the criss-cross pattern shown in FIG. 5 (Type D: A×B [B<«A]), and the zig-zag pattern also shows in FIG. 5 (Type E) leads to an increase in the surface area of the sensor compared to the Type C (and Type B) substrates.

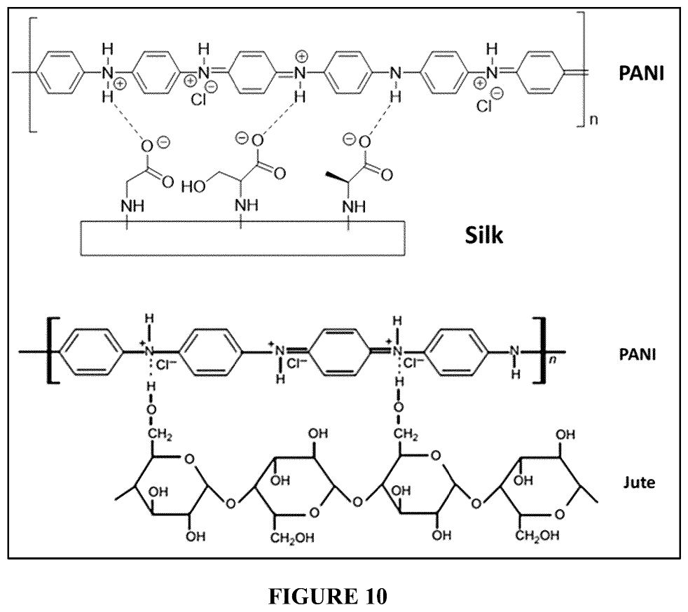

FIG. 10 diagrammatically depicts the chemical interaction between polyaniline with the natural fibres silk (upper diagram) and cellulose, i.e. jute, (lower diagrarm).

A potential issue for the 1D substrate is that its cross-sectional area, which is much lower than that of the 2D sensor, significantly reduces the capture probability of the virus or biogenic material. Thus, although narrowing the width of the sensor increases the sensitivity and decreases the response time, the droplet-capturing cross-section is compromised. Thus, in making an effective 1D sensor for virus and biogenic material, the width of the sensor should be optimized according to the conditions under which the sensor is to be used.

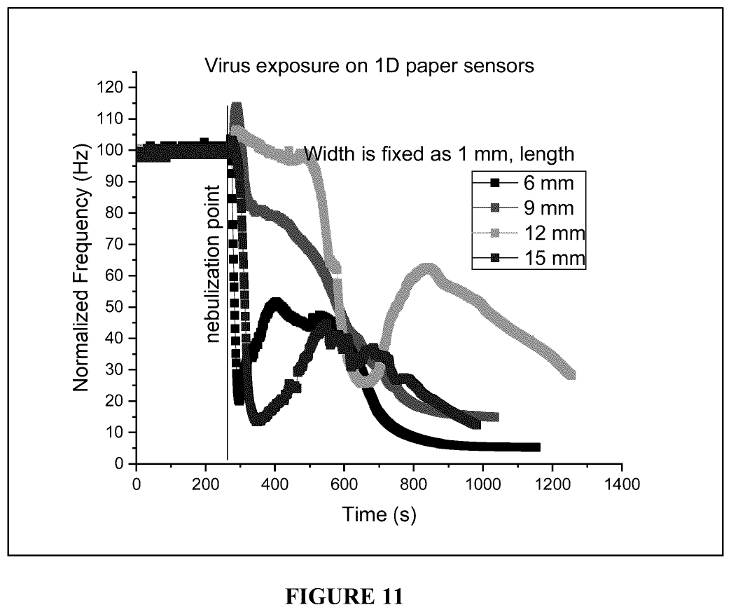

FIG. 11 depicts representative data showing the effect of length variation for the 1D paper sensor fabricated by in-situ coating of PANI on 300 gsm paper. The width of the sensor is fixed as 1 mm, but the length has been varied from 6 mm to 15 mm.

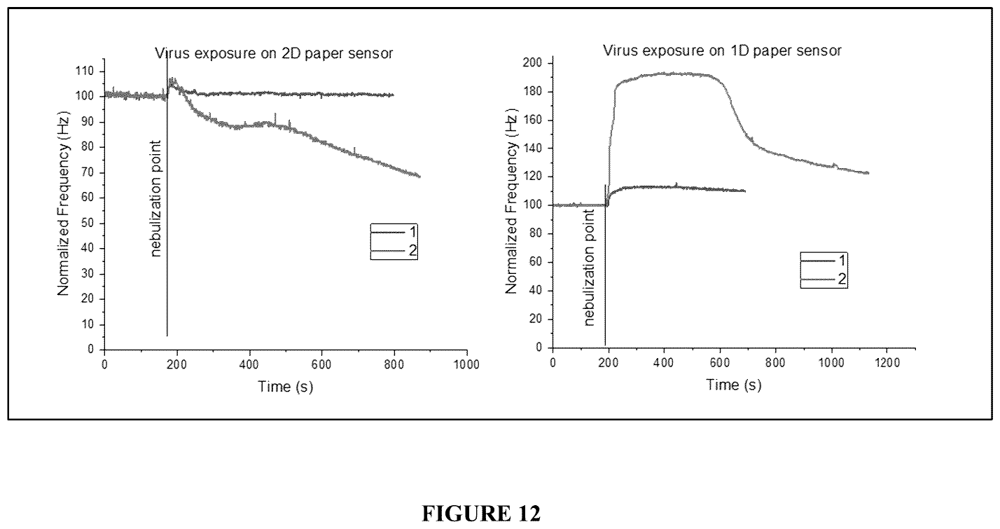

FIG. 12 depicts representative data showing a side-by-side comparison of a 2D paper-based sensor (on the left) and a 1D paper paper-based sensor (on the right) upon exposure to the same number of virus particles.

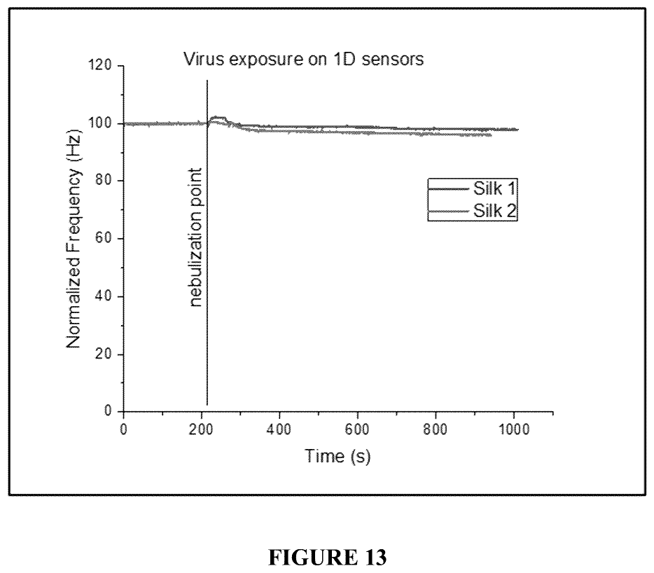

FIG. 13 depicts representative data representing the response of a 1D sensor based on silk fibres.

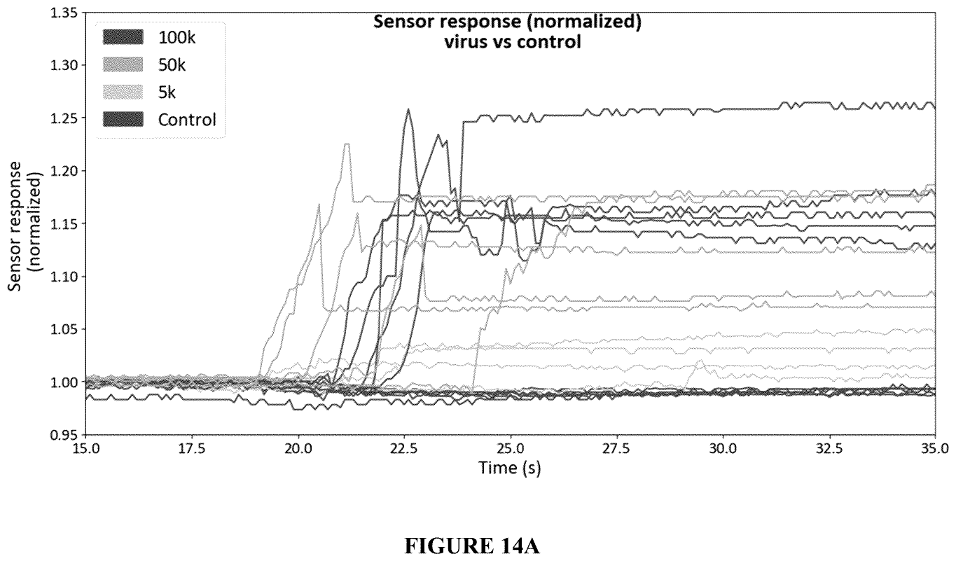

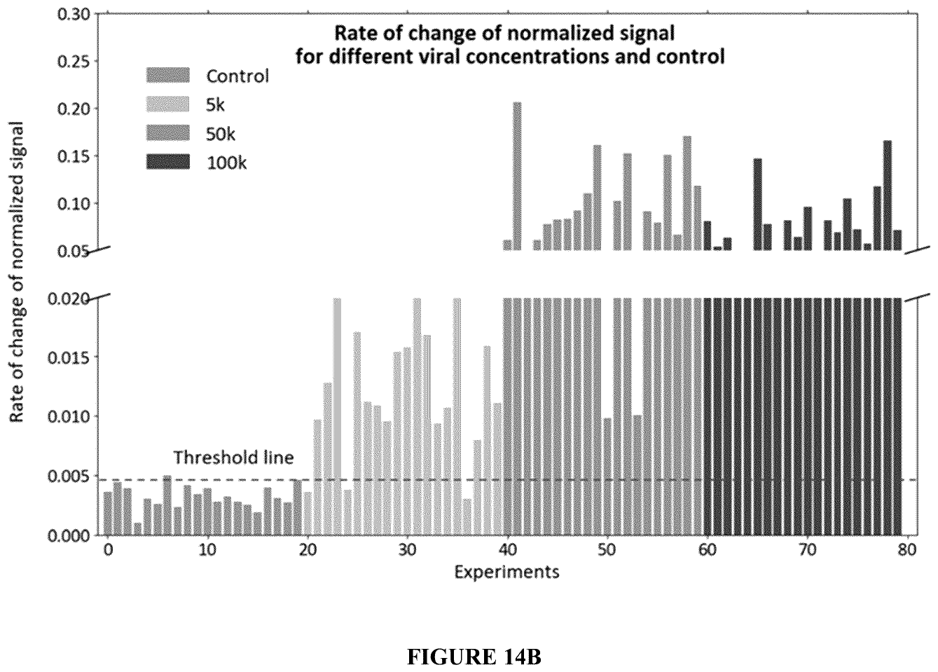

FIG. 14A is a plot of normalized sensor responses with respect to time. Responses of different concentrations of SARS Cov-2 viruses, 100 k copies (red), 50 k copies (orange) and 5 k copies (yellow) per μl of AS (artificial saliva) and control (only AS, green) are shown. A one-μl drop of these dispersions has been added around the 20th second of the scan.

FIG. 14B depicts statistics of the rate of change of the normalized responses. The change is defined as the difference between responses before the addition of the drop; and after the addition of the drop, once the signal stabilizes around the 30^{th} second, calculated from the above data. For each concentration and control set, 20 experiments have been carried out. The data of FIG. 14B shows that the rate of change for the control is below a threshold, i.e., 0.005, while the rates of change in the presence of the virus samples are greater than the threshold.

Although the present solution has been illustrated and described with respect to one or more implementations, equivalent alterations and modifications will occur to others skilled in the art upon the reading and understanding of this specification and the annexed drawings. In addition, while a particular feature of the present solution may have been disclosed with respect to only one of several implementations, such feature may be combined with one or more other features of the other implementations as may be desired and advantageous for any given or particular application. Thus, the breadth and scope of the present solution should not be limited by any of the above described embodiments. Rather, the scope of the present solution should be defined in accordance with the following claims and their equivalents.

Although the present solution has been illustrated and described with respect to one or more implementations, equivalent alterations and modifications will occur to others skilled in the art upon the reading and understanding of this specification and the annexed drawings. In addition, while a particular feature of the present solution may have been disclosed with respect to only one of several implementations, such feature may be combined with one or more other features of the other implementations as may be desired and advantageous for any given or particular application. Thus, the breadth and scope of the present solution should not be limited by any of the above described embodiments. Rather, the scope of the present solution should be defined in accordance with the following claims and their equivalents.

We claim:

1. A sensor for detecting the presence of a pathogen, the sensor comprising a one-dimensional substrate.

2. The sensor of claim 1, further comprising a coating on the substrate, the coating comprising an electrically-conducting polymer.

3. The sensor of claim 2, wherein the coating comprises polyaniline.

4. The sensor of claim 2, wherein the substrate comprises cellulose.

5. The sensor of claim 2, wherein the substrate comprises a single-strand filament.

6. The sensor of claim 2, wherein the substrate comprises a natural fibre.

7. The sensor of claim 6, wherein the substrate comprises one of silk thread and jute.

8. The sensor of claim 2, wherein the substrate has a width of less than about 25 microns.

9. The sensor of claim 8, wherein the substrate has a width of about 3 microns to about 25 microns.

10. The sensor of claim 9, wherein the substrate has a width of about 20 microns to about 25 microns.

11. The sensor of claim 2, wherein the substrate has a linear configuration.

12. The sensor of claim 2, wherein the substrate has a zig-zag configuration.

13. The sensor of claim 2, wherein the substrate has a criss-cross configuration.

14. The sensor of claim 2, wherein the substrate comprises paper.

15. The sensor of claim 14, wherein the substrate comprises 300 grams per square meter paper.

16. A method for detecting the presence of airborne pathogens, comprising:
providing a one-dimensional sensor; and
measuring a change of resistance of the sensor in response to exposure to the pathogens.

17. The method of claim 16, wherein providing a one-dimensional sensor comprises providing a sensor comprising a substrate having a width of about 25 microns or less, and a coating on the substrate, the coating comprising an electrically-conducting polymer.

18. A sensor for detecting the presence of a pathogen, comprising:
a substrate having a width of about 25 microns or less; and
a coating on the substrate, the coating comprising an electrically-conducting polymer.

19. The sensor of claim 18, wherein the coating comprises polyaniline.

20. A pathogen detection system comprising:
a sensor; and
an RC coupling circuit electrically coupled to the sensor and configured to indicate a change of resistance of the sensor based on an output frequency of the RC coupling circuit.
